(19) United States
(12) Patent Application Publication
(10) Pub. No.: US 2022/0378861 A1
(43) Pub. Date: Dec. 1, 2022

(54) COMPOSITIONS AND METHODS FOR DELIVERING CANNABINOIDS USING MICRONEEDLE DEVICES TO THE SKIN

(71) Applicant: AQUAVIT PHARMACEUTICALS, INC., New York, NY (US)

(72) Inventor: Sobin Chang, New York, NY (US)

(21) Appl. No.: 17/769,995

(22) PCT Filed: Oct. 19, 2020
(86) PCT No.: PCT/US2020/056379

§ 371 Date: Apr. 18, 2022

Related U.S. Application Data
(60) Provisional application No. 62/916,424, filed on Oct. 17, 2019.

(51) Int. Cl. A61K 36/185; A61K 9/00; A61M 5/158; A61M 5/142; A61K 31/282

(52) U.S. Cl. CPC A61K 36/185; A61K 9/0021; A61M 5/158; A61M 5/142; A61K 31/282; A61M 2037/0023

(57) ABSTRACT

The present invention provides a method for treating a disease or condition in a subject, comprising administering to the subject's skin a composition comprising an effective amount of one or more cannabinoids, wherein the composition is administered with a microneedle delivery device.

### CROSS-REFERENCE TO RELATED APPLICATIONS

This application claims the benefit of U.S. Provisional Application No. 62/916,424, filed Oct. 17, 2019, the contents of which are incorporated by reference herein in its entirety.

### FIELD OF THE INVENTION

The field of the invention relates generally to the field of medicine, specifically methods and compositions useful for treating diseases or conditions, e.g., of the skin, by delivering one or more cannabinoids.

### BACKGROUND OF THE INVENTION

Cannabis contains more than 460 compounds of which around 70 are considered as phytocannabinoids. Hempseed oil also contains the phytcannabinoid cannabidiol (CBD). Cannabis-based medications have been intensely studied since the endogenous cannabinoid system was discovered two decades ago. Cannabis-based medications exert their effects mainly through the activation of cannabinoid receptors CB1 and CB2. Cannabinoids produce numerous therapeutic effects. They have antispastic, analgesic, antiemetic, neuroprotective, and anti-inflammatory actions. They are an effective treatment against certain psychiatric diseases.

Cannabinoids have been found to have antioxidant properties, unrelated to NMDA receptor antagonism. This makes cannabinoids useful in the treatment and prophylaxis of wide variety of oxidation associated diseases, such as ischemic, age-related, inflammatory and autoimmune diseases.

Cannabinoids have been found to have particular application as neuroprotectants, for example in limiting neurological damage following ischemic insults, such as stroke and trauma, or in the treatment of neurodegenerative diseases, such as Alzheimer's disease, Parkinson's disease and HIV dementia.

Emerging clinical applications for cannabinoid therapies include Alzheimer's Disease, Amyotrophic Lateral Sclerosis (ALS), atherosclerosis, chronic pain, Diabetes mellitus, dystonia, epilepsy, fibromyalgia, gastrointestinal disorders, gliomas, cancer, Hepatitis C, Human Immunodeficiency Virus (HIV), Huntington Disease hypertension, incontinence, methicillin-resistant Staphyloccus aureus (MRSA), multiple sclerosis, osteoporosis, post-traumatic stress disorders (PTSD), pruritus, rheumatoid arthritis, sleep apnea and Tourette Syndrome.

One of the primary adverse effects of cannabinoid therapies in humans is disruption of short-term memory. That is consistent with the abundance of CB1 receptors in the hippocampus, the brain region most closely associated with memory. Cannabinoids impinge on the central nervous system by attaching to brain's neurons and interfering with normal communication between the neurons. These nerves respond by altering their initial behavior.

The phytocannabinoids in cannabis may produce adverse cardiovascular effects. A consistent effect from the phytocannabinoids in cannabis is increased heart rate. They can reduce the level of exercise which can be tolerated before the onset of angina. Cannabinoids produce profound coronary and cerebral vasodilatation in vivo by activation of vascular cannabinoid CB1 receptors. Their prominent, predictable effects on the heart, including increased work-load, increased plasma volume and postural hypotension that can impose threats to the individuals' hypertension, cerebrovascular disease or coronary arteriosclerosis. High doses of cannabis measured as 15 mg of THC are shown to increase heart rate, gross motor disturbances, and can lead to panic attacks.

Cannabinoids also produce a tolerance. Prolonged exposure to phyto synthetic or endogenous cannabinoid agonists is associated with the development of tolerance for most of their pharmacological effects essentially due to adaptive down-regulation and desensitization of cannabinoid receptors.

Currently employed methods of delivery of cannabis derived cannabinoids include inhalation delivery methods of smoking, vaporization and aerosols; oral ingestion delivery methods into the GI tract of infused products, edibles, extract oils, tinctures and soft gel caps; and intraoral delivery methods to the oral mucosa via sprays and drops of cannabis as tinctures, extracts, and emulsion compositions, and cannabis containing chewing gums.

Methods of delivering hempseed derived cannabinoids include oral ingestion delivery into the GI tract of infused products, edibles extract oils, tinctures and soft gel caps and intraoral delivery to the oral mucosa via sprays and drops of cannabis as tinctures, extracts, and emulsion compositions, and chewing gums.

Inhalation delivery methods of smoking and vaporization have no reliable dosage as medicine. Bioavailability following the smoking route was reported as 2-56%, due in part to intra- and inter-subject variability in smoking dynamics, which contributes to uncertainty in dose delivery. The number, duration, and spacing of puffs, hold time, and inhalation volume, or smoking topography, greatly influences the degree of exposure and blood levels.

Oral delivery methods of ingesting extracts, infusions and edibles forms have typically a delay in the onset of their actions making it extremely difficult in ingest the correct dosage of cannabinoids. The oral absorption of THC and CBD are typically reported as 6% bioavailability to the systemic circulation after extensive first pas liver metabolism. Oral delivery is slow and unpredictable, with peak concentrations occurring 1-5 hours post dose.

Several factors account for the low oral bioavailability of cannabinoid as compared to intravenous administration. They include low solubility and dissolution, variable absorption, degradation in the stomach, and significant first-pass metabolism to active and inactive metabolites in the liver. There may be variation in potency of cannabinoid constituents from crop to crop and even in the same cannabis depending upon its, age, moisture content and methods of curing. Furthermore oral ingested products often lack accurate information of the cannabinoid content per dosage and an accurate and reliable method to regulate the dosage of cannabinoids administered.

### SUMMARY OF THE INVENTION

It is to be understood that both the foregoing general description of the embodiments and the following detailed description are exemplary, and thus do not restrict the scope of the embodiments.

In one aspect, the invention provides methods and compositions that provide enhanced cannabinoid bioactivity, increased therapeutic activity, at lower doses and with fewer adverse actions; deliver standardized precision-metered dosage forms of cannabinoids; and administration to the skin by a more effective method of delivery through microneedle devices, making cannabinoid drug treatments more efficacious and available to a larger number of patients.

In another aspect, the invention provides method for treating a disease or condition in a subject, comprising administering to the subject's skin a composition comprising an effective amount of one or more cannabinoids, wherein the composition is administered with a microneedle delivery device.

In another aspect, the invention provides a microneedle delivery device comprising an effective amount of one or more cannabinoids for use in the methods herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is a view of a handheld microneedle injection apparatus. The syringe ejection volume is automatically controlled and dispenses into an interchangeable head containing one or several needles. The diagram shows the connection of corrugated connector and microneedle head. The rubber based connector is such that its flexibility will allow connections with small openings (1) and large ones (2) to fit and seal the microneedle head. The corrugated connector, also made of rubber (3), will further allow larger embodiments to connect to this system with the spring plate microneedle head (4).

FIG. 2 is an image of a microneedle head piece.

FIG. 3 is a schematic representation of a device in a syringe configuration. Alternative configurations include vial- and capsule-loaded configurations. The device holds a syringe (2) for automatic injection via a plurality of microneedles in the microneedle head. Ejection volume is controlled by an information processor (9). Other elements are noted: the motor or actuator (4) to control the piston (3), exchangeable and controllable needle head (1) and cam system and dial to adjust needle injection depth (5), and needle head ejector (10). Information is shown to the user in a display panel that may include a manual or touchscreen control panel (12) and data is stored in a storage unit (11) that may be removable. The needle head (1) may be controlled by an actuator (13).

FIG. 4 provide three additional views of a microneedle device. Microneedle components: (A) microneedles, (B) housing of the needles and (C) a reservoir.

FIG. 5 provides an exemplary microneedle drug delivery device.

FIG. 6 provides an exemplary microneedle drug delivery device.

FIG. 7 provides internal assembly of parts of the device of FIG. 6.

FIG. 8 provides an external push assembly view of the device of FIG. 6.

FIG. 9 provides a view of the assembled internal parts of FIG. 6.

FIG. 10 provides a view of the assembled internal parts of FIG. 6.

FIG. 11 provides a view of the device of FIG. 6.

FIG. 12 provides a view of the device of FIG. 6.

FIG. 13 illustrates a multi chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir, and can be administered on a subject. The microchannel head facilitates movement from reservoir to the subject's skin.

FIG. 14 illustrates a multi chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir, and can be administered on a subject. The microchannel head facilitates movement from reservoir to the subject's skin.

FIG. 15 illustrates a modular multi chamber microneedle drug delivery device design. This allows the chambers and the reservoir with the microneedle head to be detachable. The chambers can be replaced or substituted. It features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir and can be administered on a subject. The microchannel head facilitates movement from reservoir to the subject's skin.

FIG. 16 illustrates a multi chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed by a gravity-driven motion by shaking the device. After this the bioactive composition transfers to the reservoir and can be administered on a subject. The microchannel head facilitates movement from reservoir to the subject's skin. It also features a blender that can be activated by the subject through an external button/switch. This blender helps in mixing the bioactive composition.

FIG. 17 illustrates a multi chamber microneedle drug delivery device design that features multiple pusher that is activated individually or together by the subject. Each pusher pierces the layer separating the two chambers thereby allowing flow of bioactive composition from one chamber to another. After this, the bioactive compositions are mixed by a gravity-driven motion by shaking the device. After this the bioactive composition transfers to the reservoir and can be administered on a subject. The microchannel head facilitates movement from reservoir to the subject's skin. Each of these chambers can contain different compositions.

FIG. 18 illustrates a modular multi chamber microneedle drug delivery device design that features multiple chambers that can be attached to each other. Each chamber features a pusher that pierces the layer separating the two chambers thereby allowing flow of bioactive composition from one chamber to another. After this, the bioactive compositions are mixed by a gravity-driven motion by shaking the device. After this the bioactive composition transfers to the reservoir, and can be administered on a subject. The microchannel head facilitates movement from reservoir to the subject's skin. Each of these chambers can contain different compositions.

FIG. 19 illustrates a modular multi chamber microneedle drug delivery device design that features two chambers that can be attached to each other wherein one chamber contains the pusher that pierces the other chamber. The pusher pierces the outer layer of the attached chamber thereby allowing flow of bioactive composition from one chamber to another. After this, the bioactive compositions are mixed by a gravity-driven motion by shaking the device. After this the bioactive composition transfers to the reservoir and can be administered on a subject. The microchannel head facilitates movement from reservoir to the subject's skin. Each of these chambers can contain different compositions.

FIG. 20 illustrates a microchannel head adapter that can be used with regular syringes. It comes with a cap that covers the microchannel head.

### DETAILED DESCRIPTION OF THE INVENTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

It is contemplated that any method or composition described herein can be implemented with respect to any other method or composition described herein.

As used herein, the term “about” means plus or minus 10% of the numerical value of the number with which it is being used.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A. B, and C; A, B. or C; A or C; A or B; B or C; A and C; A and B; B and C; A (alone), B (alone); and C (alone). In one embodiment, the invention provides a method for administering one or more cannabinoids to a subject, comprising administering to the subject's skin a composition comprising an effective amount of one or more cannabinoids, wherein the composition is administered with a microneedle delivery device.

In another embodiment, the invention provides a method for treating a disease or condition in a subject, comprising administering to the subject's skin a composition comprising an effective amount of one or more cannabinoids, wherein the composition is administered with a microneedle delivery device.

In another embodiment, the invention provides a method of alleviating pain or reducing undesirable side effects associated with radiation therapy or chemotherapy in a subject in need thereof comprising administering to the subject's skin a composition comprising an effective amount of one or more cannabinoids, wherein the composition is administered with a microneedle delivery device.

In another embodiment, the invention provides a method of treating anxiety in a subject in need thereof comprising administering to the subject's skin a composition comprising an effective amount of one or more cannabinoids, wherein the composition is administered with a microneedle delivery device.

In another embodiment, the invention provides a method of treating depression in a subject in need thereof comprising administering to the subject's skin a composition comprising an effective amount of one or more cannabinoids, wherein the composition is administered with a microneedle delivery device.

In another embodiment, the invention provides a method of treating nausea and/or vomiting in a subject in need thereof comprising administering to the subject's skin a composition comprising an effective amount of one or more cannabinoids, wherein the composition is administered with a microneedle delivery device.

In another embodiment, the invention provides a method of lowering blood pressure in a subject in need thereof comprising administering to the subject's skin a composition comprising an effective amount of one or more cannabinoids, wherein the composition is administered with a microneedle delivery device.

In another embodiment, the invention provides a method of treating acne in a subject, comprising administering to the subject's skin a composition comprising an effective amount of one or more cannabinoids, wherein the composition is administered with a microneedle delivery device. In some embodiments, the cannabinoid comprises cannabidiol. In accordance with certain embodiments, the composition is preferably administered to an area of the skin which is affected by acne. Preferably, the application of cannabinoid in accordance with certain embodiments results in reduction in redness, itch, pain or irritation, a reduction in pimples, papules, blisters or pustules, a reduction in infection, less breakdown and loss of collagen and elastin in the skin, a reduction of swelling, cracking, weeping, crusting, and scaling and/or a general decrease in inflammation.

In another embodiment, the invention provides a method of treating inflammation (local and/or systemic) in a subject, comprising administering to the subject's skin a composition comprising an effective amount of one or more cannabinoids, wherein the composition is administered with a microneedle delivery device.

In another embodiment, the invention provides a method of treating pain (localized and/or systemic) in a subject, comprising administering to the subject's skin a composition comprising an effective amount of one or more cannabinoids, wherein the composition is administered with a microneedle delivery device. In some embodiments, the body part of the subject that is treated is selected from an arm, leg, knee, torso, head, neck, foot as well as those parts that make-up them) and combinations thereof. In some embodiments, the treatment of the pain is localized pain.

Non-limiting examples of injuries or other physical diseases or conditions causing pain and/or inflammation for which embodiments of the present disclosure can be used to reduce or lessen the pain and/or inflammation can include arthritis conditions (e.g., osteo, rheumatoid, psoriatic, fibromyalgia, etc.), head pain (e.g., concussion, head ache, migraine), orthopedic injuries or conditions (e.g., bone fracture or break; dislocated joint or bone; torn, stressed or strained ligament or tendon; bruising or trauma to tissue; back or spinal pain or herniated disc; tendonitis; gout, bursitis), muscles aches and pains (e.g., from stress and physical exertion) and post-surgery recovery (e.g., recovery from orthopedic surgery to repair a broken bone, back condition such as herniated disk or torn ligament, orthoscopic surgery.

The one or more cannabinoids that can be administered is not particularly limiting. In some embodiments, the one or more cannabinoids is selected from cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and mixtures of cannabinoids. The cannabinoids can be natural or synthetically produced. The cannabinoids can also be derived from crude or purified extracts from cannabis (e.g., Cannabis sativa). Mixtures of natural and synthetically produced cannabinoids can also be used. In some embodiments, a combination of cannabinoids is administered comprising CBD and CBG.

In some embodiments, the cannabinoid is selected from Δ9-tetrahydrocannabinol (Δ9-THC), Δ8-tetrahydrocannabinol (Δ8-THC), and Δ9-tetrahydrocannabinolic acid (THCa).

As used herein, “treat” and all its forms and tenses (including, for example, treating, treated, and treatment) refers to therapeutic and prophylactic treatment. In certain aspects of the invention, those in need of treatment include those already with a pathological disease or condition of the invention, in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the disease or pathological condition. The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition. The terms “effective amount” or “therapeutically effective amount” or “therapeutic effect” refer to an amount of a cannabinoid composition to “treat” a disease, condition or disorder in a subject such as, a mammal. In the case of any disease or condition, the therapeutically effective amount of an agent has a therapeutic effect and as such can enhance or boost response against diseases or condition, such as, for example, Alzheimer's Disease, Amyotrophic Lateral Sclerosis (ALS), atherosclerosis, chronic pain, Diabetes mellitus, dystonia, epilepsy, fibromyalgia, gastrointestinal disorders, gliomas, cancer, Hepatitis C, Human Immunodeficiency Virus (HIV), HIV dementia, Huntington Disease hypertension, incontinence, methicillin-resistant Staphylococcus aureus (MRSA), multiple sclerosis, osteoporosis, post-traumatic stress disorders (PTSD), pruritus, rheumatoid arthritis, sleep apnea and Tourette Syndrome; increase heart rate, produce coronary and cerebral vasodilation, increate plasma volume and postural hypotension; disrupt, inhibit or stop short-term memory; reduce morbidity and mortality; improve quality of life; or a combination of such effects.

In some embodiments, the dose of the one or more cannabinoids administered ranges from about 0.1 mg to about 1000 mg, from about 0.1 mg to amount 100 mg, from about 0.5 mg to about 50 mg, from about 1.0 mg to about 25 mg, or from about 1.0 mg to about 10 mg.

In some embodiments, the composition comprising the one or more cannabinoids administered contains between 2.5% and 5% cannabinoid by weight. In some embodiments, the composition administered comprises between 20 mg and 100 mg cannabinoid. In some embodiments, the composition is administered one or two times per day. In some embodiments, the total daily dose applied to the skin is between 20 mg and 200 mg cannabinoid.

The amount of the therapeutic agents of the invention which will be effective in promoting a therapeutic effect can be determined by standard clinical techniques. The precise dose to be employed in the formulation will also depend on the judgment of the practitioner and each subject's circumstances. Effective doses can be extrapolated from dose-response curves derived from in vitro or animal model test systems.

One skilled in the art can readily determine an appropriate dosage regimen for administering cannabinoids of the invention to a given subject. For example, the compound(s) or composition(s) can be administered to the subject in one administration or multiple administrations. Where a dosage regimen comprises multiple administrations, it is understood that the effective amount of the compound(s) or composition(s) administered to the subject can comprise the total amount of the compound(s) or composition(s) administered over the entire dosage regimen. The exact amount will depend on the purpose of the treatment, the subject to be treated, and will be ascertainable by a person skilled in the art using known methods and techniques for determining effective doses. In some embodiments, the amount of the therapeutic agent that can be administered includes between about 0.1 μg/kg to about 100 mg/kg. In some embodiments, the amount of the one or more cannabinoids that can be administered includes between about 1.0 μg/kg to about 10 mg/kg.

The therapeutic compositions can be administered one time or more than one time, for example, more than once per day, daily, weekly, monthly, or annually. The duration of treatment is not particularly limiting. The duration of administration of the therapeutic composition can vary for each individual to be treated/administered depending on the individual cases and the diseases or conditions to be treated. In some embodiments, the therapeutic composition can be administered continuously for a period of several days, weeks, months, or years of treatment or can be intermittently administered where the individual is administered the therapeutic composition for a period of time, followed by a period of time where they are not treated, and then a period of time where treatment resumes as needed to treat the disease or condition. For example, in some embodiments, the individual to be treated is administered the therapeutic composition of the invention daily, every other day, every three days, every four days, 2 days per week 3 days per week, 4 days per week, 5 days per week or 7 days per week. In some embodiments, the individual is administered the therapeutic composition for 1 week, 2 weeks, 3 weeks, 4 weeks, 1 month, 2 months, 3 months, 4 months, 5 months, 6 months, 7 months, 8 months, 9 months, 10 months, 11 months, 1 year, or longer.

The term “subject” as used herein is not limiting and is used interchangeably with patient. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like.

The term “disease or condition,” as used herein is not limiting and can include any disease or condition that can be treated with cannabinoids. In some embodiments, the subject administered does not have a disease or condition, and administers the cannabinoid composition for recreational purpose. In some embodiments, the disease or condition is selected from anorexia, emesis, sunburn, photodamaged skin, acne, psoriasis, atopic dermatitis, pain, inflammation, multiple sclerosis, neurodegenerative disorders (e.g., Parkinson's disease, Huntington's disease, Tourette's syndrome, Alzheimer's disease), epilepsy, glaucoma, osteoporosis, schizophrenia, cardiovascular disorders, cancer, actinic keratosis, obesity, and metabolic syndrome-related disorders.

In some embodiments, subjects treated using the methods have a compromised immune system, a cancer, a pulmonary disease or a condition that causes violent tremors.

In some embodiments, the subjects are administered the compositions for recreational and not therapeutic purposes.

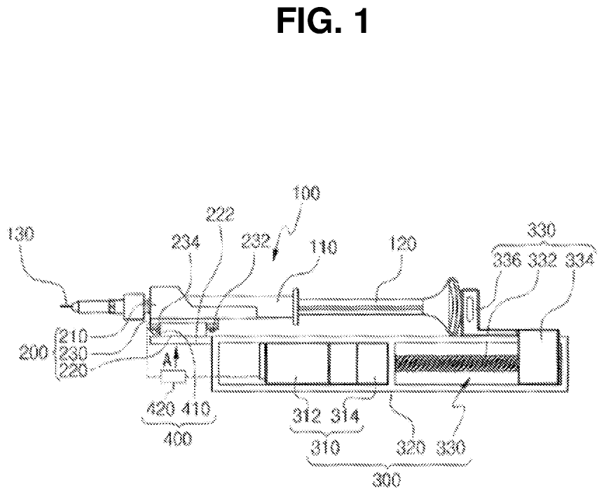

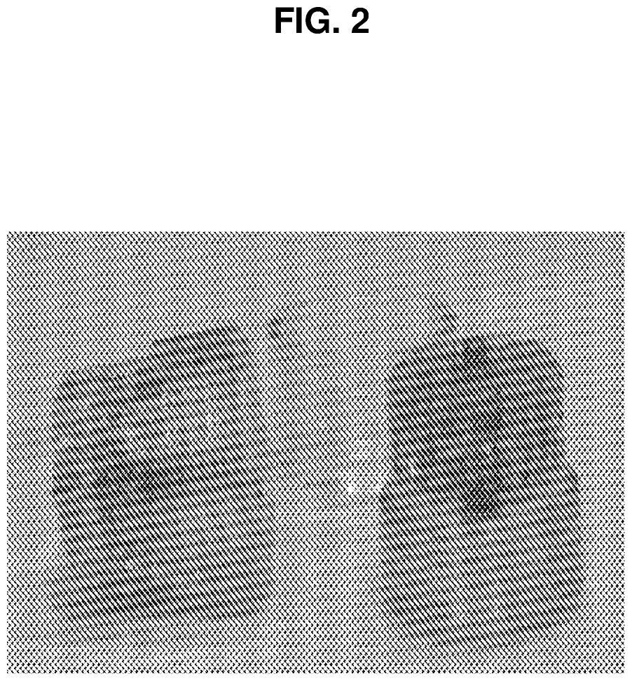

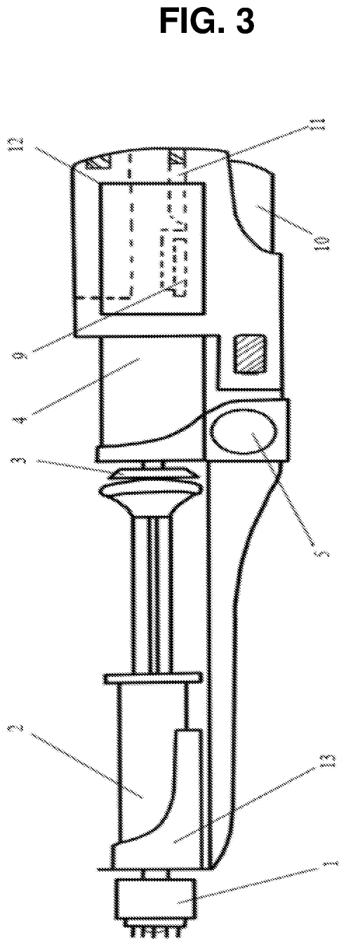

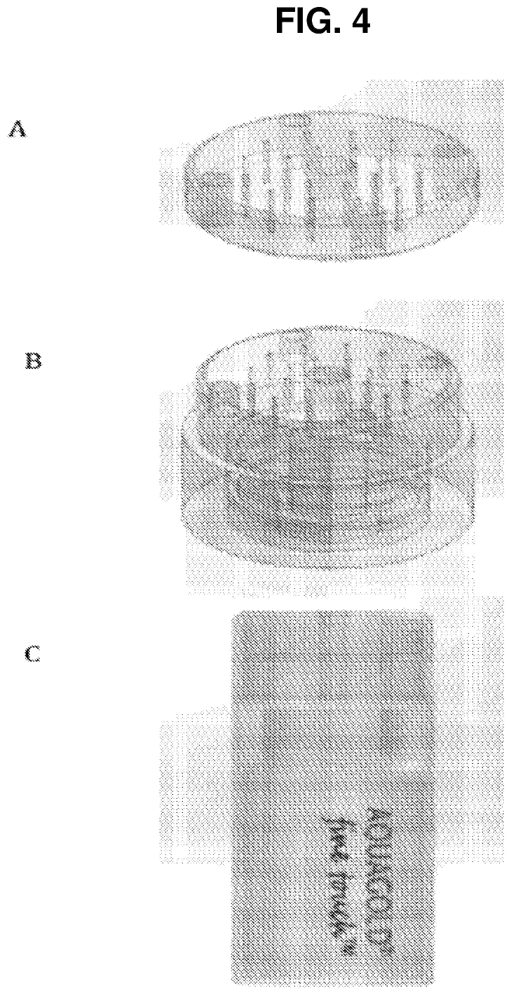

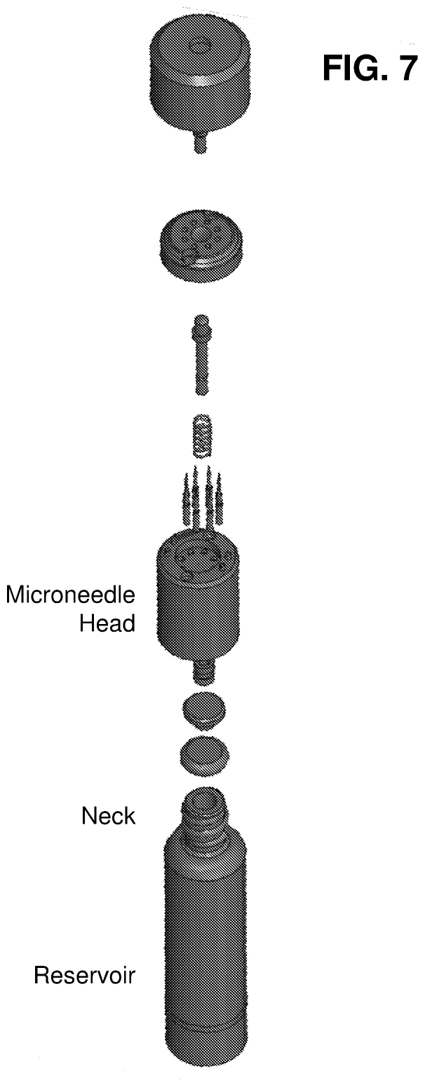

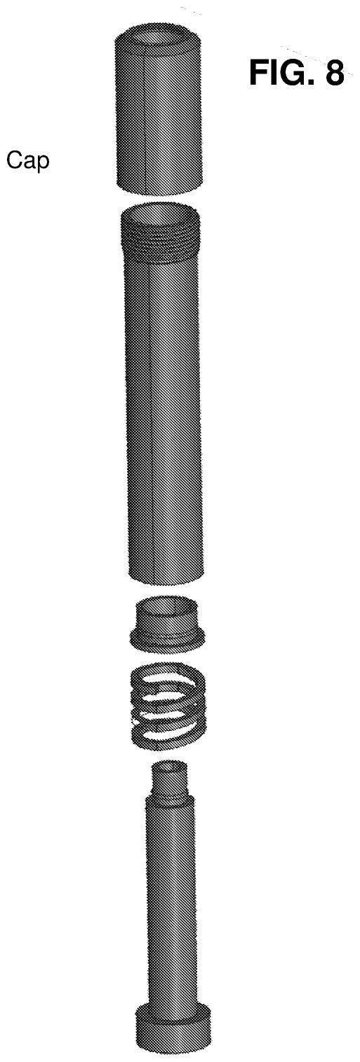

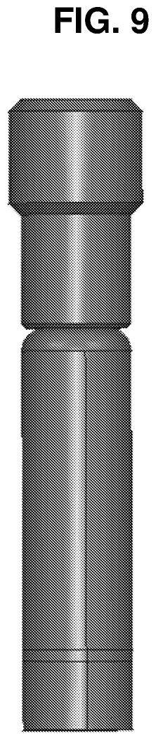

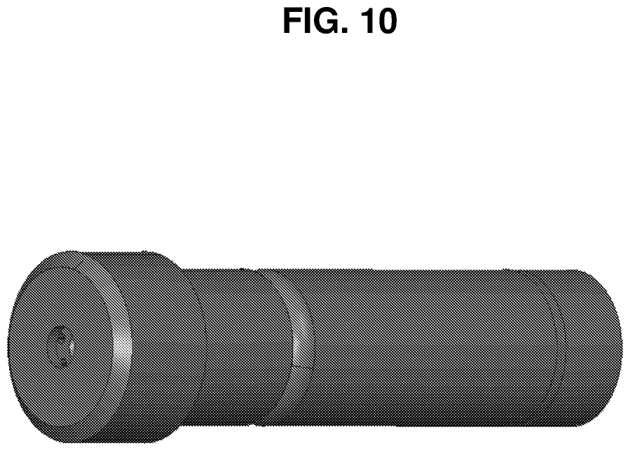

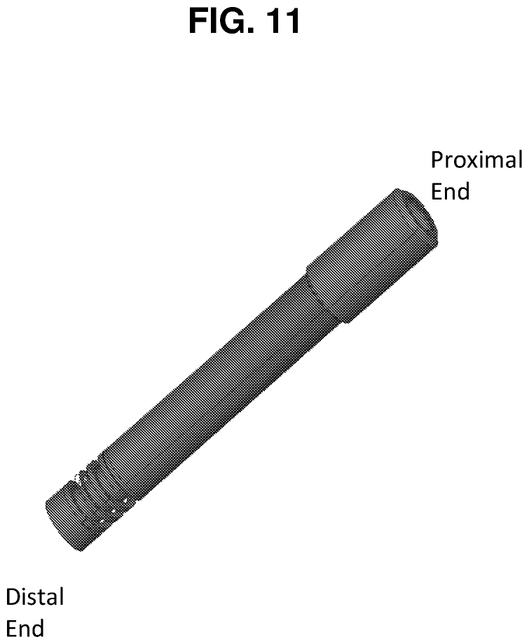

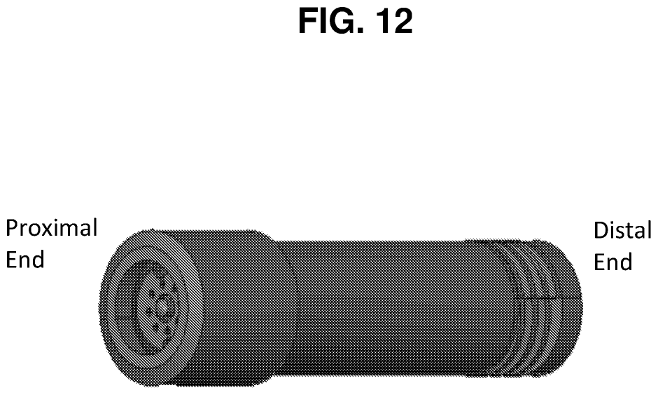

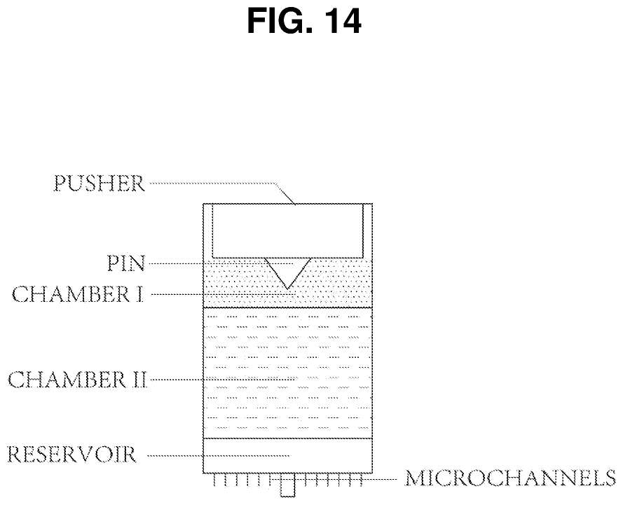

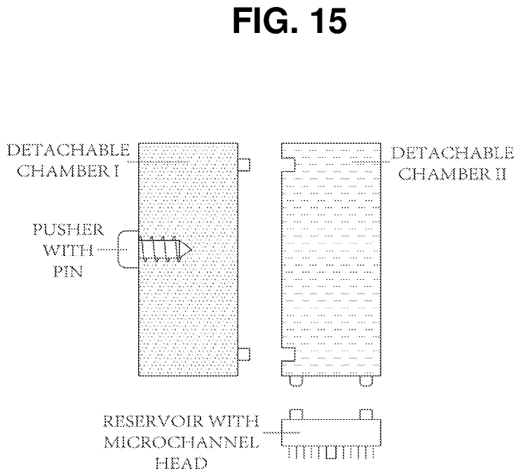

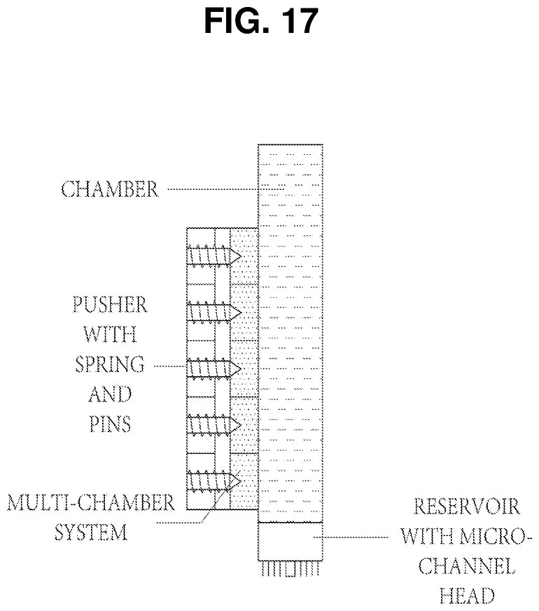

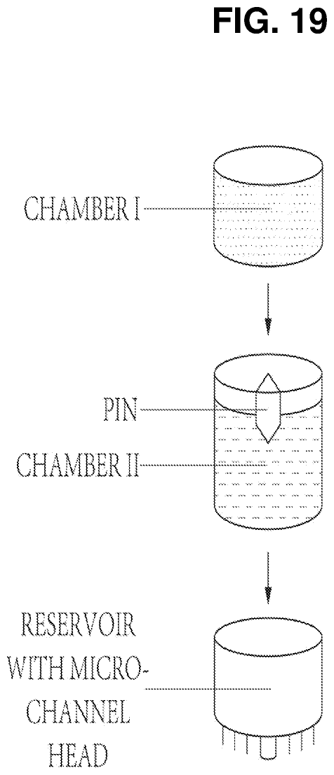

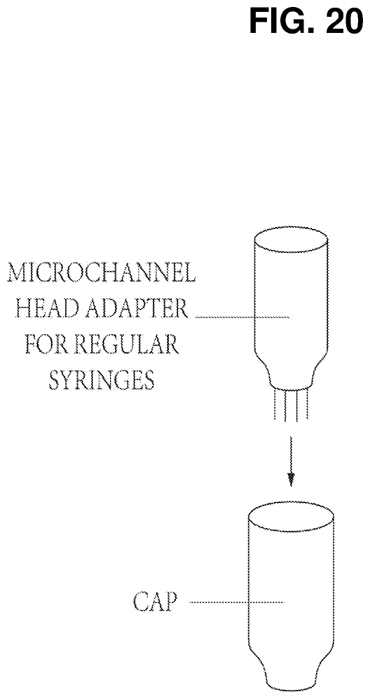

In some embodiments, the microneedle delivery device useful in the methods of the invention is depicted in any of FIGS. 1-20. As provided herein, a microneedle delivery device is used to deliver the cannabinoid composition. In some embodiments, the microneedle array can be used to deliver the cannabinoid composition directly to the dermis (the second layer of skin). In some embodiments, the microneedle devices as disclosed herein deliver the cannabinoid composition into the dermal and epidermal junction area. In another embodiment, the microneedle device does not penetrate into the dermal layer but only disrupts the superficial portion of the skin, referred to as stratum corneum.

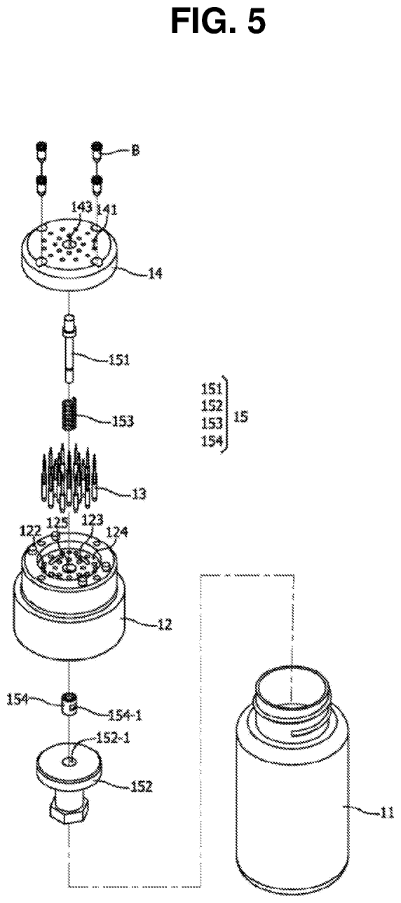

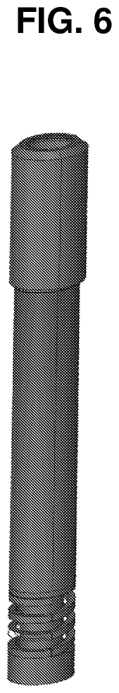

In some embodiments, the microneedle delivery device useful in the methods of the invention is depicted in FIG. 5. In some embodiments, the microneedle drug delivery device is as described in Korean Patent No. 10-1582822, which is incorporated by reference herein in its entirety. In some embodiments, the microneedle drug delivery device is as described in US Pat. Appl. Pub. No. 2016/0175408 Δ1, which is incorporated by reference herein in its entirety. In some embodiments, the microneedle device useful in the methods of the invention is depicted in any of FIGS. 6-20.

In some embodiments, the microneedle device that can be used comprises multi chambers. In some embodiments, the device comprises a plurality of modular or replaceable chambers, wherein the chambers can hold the cannabinoid compounds or compositions. In some embodiments, the cannabinoids can be combined with one or more additional agents, either in the same chamber as the cannabinoid or in a different chamber. In some embodiments, the multi-chamber device comprises one or more microneedles, wherein the microneedles are hollow or non-hollow, wherein one or multiple grooves are inset along an outer wall of the microneedles. In some embodiments, the multi-chamber device comprises a chamber that serves as a reservoir that holds the composition to be delivered, wherein the reservoir is attached to or contains a means to encourage flow of the antineoplastic composition contained in the reservoir into the skin of a subject.

In some embodiments, the chambers can hold a cannabinoid compound or composition in a powder form or in an aqueous solution.

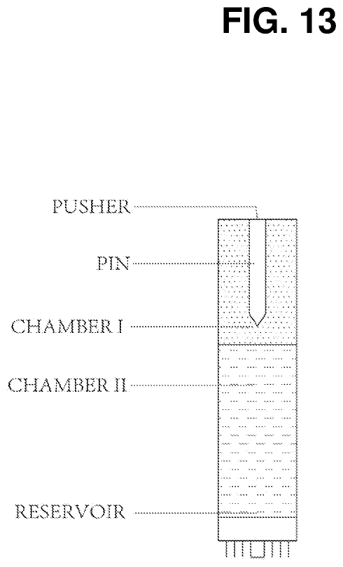

In some embodiments, the device comprises a chamber that comprises a pin that punctures another chamber to allow flow of contents from one of the chambers into the other chamber. See, for example, FIGS. 13-19.

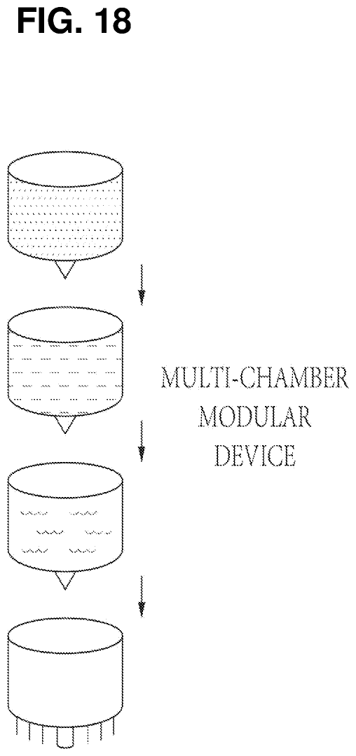

In some embodiments, the multi chamber microchannel delivery device is modular as described in FIG. 18. In some embodiments, each chamber of the device can be removed and added to the device through a push pin, mechanical or magnetic fittings.

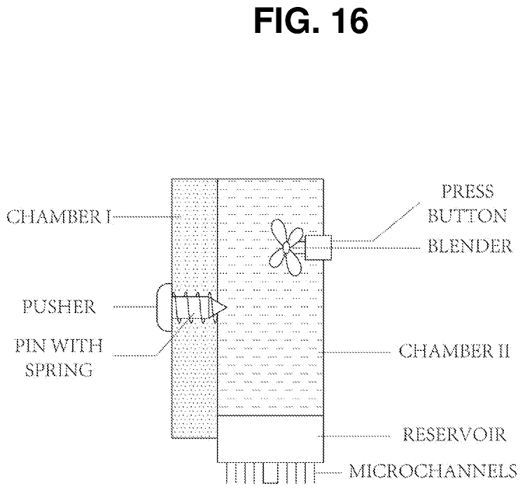

In some embodiments, the chamber contains a means for mixing the components, such as a blender element as shown in FIG. 16.

In some embodiments, the lining between the chambers are made of plastic films with low puncture resistance. In some embodiments, the lining between the chambers are made of deformable, preferably elastic material.

In some embodiments, the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the bioactive composition contained in the reservoir into the skin.

In some embodiments, the composition is administered by the microneedle delivery device with a repeated motion of penetrating the microneedle delivery device into the skin of the subject. In some embodiments, the composition is delivered into the skin by passing through the one or multiple grooves along the outer wall of the microneedle. In some embodiments, the microneedles are non-hollow.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 2.5 mm in length and from 0.01 mm to about 0.05 mm in diameter.

In some embodiments, the microneedles are made from a substance comprising gold.

In some embodiments, the one or more microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles. In some embodiments, the microneedle delivery device is repeatedly pressed against the subject's skin to deliver the composition to the area of the skin to be treated. In some embodiments, the microneedle delivery device is repeatedly pressed about 10, about 20, about 30, about 40, about 50, about 100, about 200, about 300, about 400, about 500, about 600, about 700, about 800, about 900, about 1000, about 1100, about 1200, about 1300, about 1400, about 1500, about 1600, about 1700, about 1800, about 1900, or about 2000 or more times to administer the composition.

In some embodiments, the microneedle delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrlidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microneedle delivery device is capable of delivering compositions directly to the epidermal, dermal and subcuticular layers of the skin. Therefore, it should be understood that further embodiments developed for use with non-hollow or hollow microneedle systems of delivery by those skilled in the art fall within the spirit and scope of this disclosure.

In another aspect, a microneedle device for use in the methods described herein is a device such as described in U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a seling mechanism that is secured to the tips of the microneedles. In another instance, the device may further include means for providing feedback to indicate that delivery of the material from the reservoir has been initiated or completed. An osmotic pump may be included to expel the material from the reservoir. A plurality of microneedles may be disposed at an angle other than perpendicular to the substrate. In certain instances, the at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrolidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment, a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising a cannabinoid into the skin in a process that is painless compared to injectables. Administration can result in easy and precise delivery of a composition comprising an antineoplastic agent with generally no bruising, pain, swelling and bleeding. Delivery of a cannabinoid agent may include sensitive areas and areas difficult to treat with traditional methods, such as around the eyes and mouth.

The device may include means, manual or mechanical, for compressing the reservoir, creating a vacuum, or otherwise using gravity or pressure to drive the cannabinoid from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microneedle device enhances transportation of drugs across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons.

The array may be attached to a reservoir to hold the substances to be delivered, and this reservoir may be any volume (about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics metalloid, polymer, or combination thereof, including composites, such as gold, steel, silicon, PVP (polyvinylpyrrolidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solution from the reservoir through to the dermis.

A microneedle array can consist of from about 1 to about 500 microneedles, which will be anywhere from about 0.1 to about 2.5 mm in length and from 0.01 to about 0.5 mm in diameter, and be composed of any metal, metal alloy, metalloid, polymer, or combination thereof, such as gold, steel, silicon, PVP (polyvinylpyrrolidone), etc. The microneedles can each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array can be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The delivered substances may be of varying viscosities and concentration, from 0.01% to 100%, and can be administered via the microneedle array either independently or in conjunction with the aforementioned compositions.

The reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

A cadre of microneedles housed in a plastic or polymer composite head can be used to deliver treatment solutions, directly to the dermis, the second layer of skin or the topical layer of skin. The application of a mechanical load to the pin of the spring lock system enables the micro needles to puncture the epidermal barrier and deliver the desired substances directly to the dermis for faster, more efficient, and more effective absorption by the skin. The spring plate mechanism, housed in the plastic or polymer composite cartridge, is effectively the interface whereby the manual direct application mechanism calibrates the controlled delivery of the treatment solution into the skin.

In some embodiments, the methods further comprise administering to the subject one or more additional agents or therapies. In some embodiments, the additional agent or therapy can include one or more therapies selected from radiation, surgery, chemotherapy, simple excision of cancer tissue, Mohs micrographic surgery, curettage and electrodesiccation, cryosurgery, photodynamic therapy, topical chemotherapy, and topical immunotherapy (e.g., imiquimod).

The additional therapeutic agent can be administered by any means and is not limiting. In some embodiments, the additional therapeutic agent can be administered with a microneedle delivery device, alone or in combination with the cannabinoid composition.

In some embodiments, the one or more cannabinoids are administered (together or separately) in combination with one or more therapies to treat a disease or condition in a subject, for example, one of the diseases or conditions herein.

In some embodiments, the combination therapy can include an antineoplastic agent. In some embodiments, the anti-neoplastic agent that can be used in methods of the present disclosure include, but are not limited to, e.g., alkylating agents and platinum compounds (e.g., cisplatin, carboplatin, oxaliplatin, mechlorethamine, cyclophosphamide, chlorambucil, ifosfamide), anti-metabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), cancer antigens, checkpoint inhibitors, immune cells (activated immune cells, activated lymphocytes, engineered cells), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins, and the like.

In some embodiments, the antineoplastic agent is selected from imiquimod, fluorouracil, vismodegib, 5-FU, sonidegib and combinations thereof. In some embodiments, these agents can be used in combination with one or more cannabinoids to treat basal cell carcinoma.

In some embodiments, the antineoplastic agent is cemiplimab-rwlc. In some embodiments, cemiplimab-rwlc can be used in combination with one or more cannabinoids to treat cutaneous squamous cell carcinoma.

In some embodiments, the antineoplastic agent is selected from aldesleukin, cobimetinib, dabrafenib, dacarbazine, IL-2, talimogene laherparepvec, interferon alfa-2b, ipilimumab, pembrolizumab, trametinib, nivolumab, nivolumab, peg-interferon Alfa-2b, pembrolizumab, aldesleukin, dabrafenib, trametinib, vemurafenib and combinations thereof. In some embodiments, these agents can be used in combination with one or more cannabinoids to treat melanoma.

In some embodiments, the antineoplastic agent is selected from avelumab, pembrolizumab and a combination thereof. In some embodiments, these agents can be used in combination with one or more cannabinoids to treat Merkel cell carcinoma.

In some embodiments, the antineoplastic agent is selected from vemurafenib, BRAF inhibitor and combinations thereof. In some embodiments, these agents can be used to treat malignant melanoma. In some embodiments, the antineoplastic agent for use in some embodiments is selected from LDE225B, vismodegib, RIVEDGE, PD-L1, Patidegib, Nivolumab, Nivolumab+Ipilimumab, Imiquimod, Metvix PDT, Diclofenac, Diclofenac+Calcitriol, Calcitriol, Methylaminolevulinate, Fractionated 5-aminolevulinic acid hydrochloride, PEP005, SUBA-Itraconazole, methyl-aminolevulinatem, Remetinostat, verteporfin PDT, Sonidegib, Itraconazole, vismodegib, Picato, Resiquimod, API 31510, Aminolevulinic acid, arsenic trioxide, Patidegib, REGN2810, Buparlisib, Oshadi D & Oshadi R, Celecoxib, Tazarotene, Sinecatechins 10%, aminolevulinic acid hydrochloride, Vismodegib, FOLFOX, FOLFIRI, Bevacizumab, Tazarotene, Hexylaminolevulinate, Aminolevulinic Acid, Nano Emulsion, Methylaminolevulinate, liposomal T4N5 lotion, Carboplatin, Cyclophosphamide, Etoposide, Methotrexate, Vincristine Sulfate, Acetylcysteine, Bevacizumab, Eflornithine, Celecoxib, erlotinib hydrochloride, Poly-ICLC, aminolevulinic acid, eflornithine, triamcinolone, 18F-fludeoxyglucose (18F-FDG), 18F-FPPRGD2, Fluconazole, cevimeline hydrochloride, megestrol acetate, Amifostine, Carboplatin, Etoposide, Ifosfamide, Hypericum perforatum, Docetaxel, Nicotinamide, doxepin hydrochloride, capecitabine, oxaliplatin, DetoxPC, Capecitabine, Carboplatin, epirubicin hydrochloride, cisplatin, paclitaxel, Tretinoin, doxorubicin hydrochloride, gemcitabine hydrochloride, indinavir sulfate, ritonavir, 5-fluorouracil, and combinations thereof. In some embodiments, the antineoplastic agents above can be used to treat basal cell carcinoma. In some embodiments, the antineoplastic agents above can be used to treat actinic keratosis.

In some embodiments, the antineoplastic agent is selected from Imiquimod, VDA-1102, Fluorouracil, Cetaphil, SOR007 (Uncoated Nanoparticulate Paclitaxel), Aminolevulinic Acid, PEP005 (ingenol mebutate), ingenol disoxate, Ingenol Mebutate, Aminolevulinic Acid (ALA), Biafine, Polysporin, 5-FU, A-101 Solution (High-Concentration Hydrogen Peroxide), lidamycin phosphate and benzoyl peroxide 1.2%/3.75% combination, Eflornithine, Triamcinolone, Polyphenon E, BLU-U, ACT01, Levulan®, Kerastick®, perillyl alcohol, aminolevulinic acid, liposomal T4N5, Aminolevulinic Acid, GDC 695, Diclofenac Sodium, KX2-391, ingenol disoxate, ingenol disoxate, Celecoxib, Bupivacaine+Clonidine, Secukinumab, Pimecrolimus and combinations thereof. In some embodiments, the antineoplastic agents above can be used to treat basal cell carcinoma. In some embodiments, the antineoplastic agents above can be used to treat actinic keratosis.

In some embodiments, the one or more cannabinoids are administered (together or separately) in combination with other topically applied analgesic and/or systemically available agents for the treatment of pain.

Examples of such analgesic agents include, but are not limited to: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds contemplated by as suitable in the present invention include, but are not limited to morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

In addition, other active agents may be included in the composition of the present invention, e.g., topically-effective anaesthetics such as xylocaine, cocaine, lidocaine, benzocaine, etc., which may provide a more immediate, if less effective in the long run, level of pain relief until the analgesic agent becomes fully effective.

Still other agents can also be administered, preferably topically, to potentiate the effects of the topically-administered cannabidiol. For example, dextromethorphan, a non-addictive opioid compound, can be co-administered, preferably topically, although parenteral administration is also effective, to enhance the effectiveness of the topically administered agent. Without wishing to be bound by theory, it is believed that dextromethorphan has previously unappreciated analgesic properties in peripheral nerves. Suitable concentrations of dextromethorphan are routinely ascertainable by the skilled worker, and include the normal therapeutic amounts administered parenterally for conventional purposes, e.g., as a cough suppressant, or less, and routinely determinable amounts for topical administration.

In some embodiments, the one or more cannabinoids are administered (together or separately) in combination with an agent (or combination) useful to treat acne. The agent useful to treat acne may be topically administered or systemically administered.

Examples of available agents which may be used in conjunction with the present compositions and methods for the treatment of acne include, but are not limited to: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

In one embodiment, the compositions of the present invention further comprises one or more of the following agents for the treatment of acne: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporine and cytokine synthesis inhibitors, tetracycline, minocycline, and doxycycline, or any combination thereof.

In some embodiments, the one or more cannabinoids are administered (together or separately) in combination with one or more additional agents useful to treat skin conditions and/or to improve the overall appearance or heath of skin. The compositions can be provided in freeze dried or lyophilized form and reconstituted prior to use.

In some embodiments, the one or more additional agents are selected from hyaluronic acid, one or more vitamins, collagen, one or more minerals, selenium, retinol, retinoic acid, a bleaching/whitening agent, collagen, glutathione, anthocyanidins, EPA, DHA, lecithin, CoQ10, magnesium, a neuromodulator, poly-L-lactic acid, minoxidil, platelet rich plasma, bimatoprost, an anesthetic and combinations thereof.

In some embodiments, the neuromodulator comprises botulinum toxin (e.g., botulinum toxin of serotype A, B, C, D, E, F or G). Botulinum toxin, a neurotoxic protein, is used cosmetically and therapeutically for treatment of facial lines and wrinkles, upper motor neuron syndrome, excessive sweating, cervical dystonia, chronic migraine, and overactive bladder. The toxin is generally injected into the subcutaneous muscles at the target areas, and works by temporarily (for a period of six weeks to eight months, depending on the location and the dose) inhibiting the release of acetylcholine at the neuromuscular junction and thus paralyzing the muscles achieve the desired affects (BOTOX (onabotulinumtoxinA) [prescribing information]. Irvine, Calif. Allergan, Inc. January 2013). Botulinum toxin refers to any botulinum toxin, including but not limited to botulinum toxin type A, botulinum toxin type B, botulinum toxin type Cl, botulinum toxin type D, botulinum toxin type E, botulinum toxin type F and botulinum toxin type G. Botulinum toxin type A includes, for example, Botox, Dysport and Xeomin. Botulinum toxin type B includes, for example, MyoBloc. Botulinum toxin may be provided in a liquid or powder form. A powdered form may be, for example, a sterile, lyophilized preparation. Lyophilized preparations may be reconstituted prior to application. Alternatively, botulinum toxin may be provided as a sterilized pre-dissolved solution. Botulinum toxin may be formulated in an amount of about 0.01 to about 60 units.

In some embodiments, the composition comprises one or more vitamins or minerals. The most common vitamins used today to promote skin health are A, B, C, D, and E, while the most common minerals used include zinc and calcium. When referring to a vitamin, it would be understood that all chemical forms of the vitamin are contemplated.

B vitamins are a group of water-soluble vitamins that play important roles in cell metabolism. The B vitamins are B1 (thiamine), B2 (riboflavin), B3 (niacin), B5 (pantothenic acid), B6 (pyridoxine), B7 (biotin), B12 (cobalamin) and folic acid. The B vitamins play an important role in many aspects of the body's functioning, and a vitamin B deficiency can have a serious impact on overall health.

In some embodiments, the bleaching/whitening agents that may be used in the compositions described herein include, but are not limited to, hydroquinone, kojic acid, ascorbic acid, magnesium ascorbyl phosphate or ascorbyl glucosamine, hydroquinone, licorice extract (e.g., Glycyrrhiza Glabra (licorice) root extract), an alpha MSH antagonist (e.g. undecylenoyl phenylalanine), phytic acid, monobenzyl ether of hydroquinone, azelaic acid, kojic acid, mequinol, retinoids (e.g., tretinoin, adapalene), soy proteins, alpha-hydroxy acids (e.g., glycolic acid), trichloroacetic acid, salicylic acid, hydroquinone-beta-D-glucopyranoside, paper mulberry, glabridin, 4-isopropylcetchol, aleosin, N-acetyl-4-S-cycteaminylphenol, N-propionyl-4-S-cysteaminylphenol, N-acetyl glucosamine, tranexaminc acid and mixtures thereof.

Also provided herein are compositions comprising one or more cannabinoids for use in the methods of the invention. In some embodiments, the compositions comprise one or more additional active agents as provided herein. In some embodiments, the compositions are formulated as pharmaceutical composition comprising pharmaceutically acceptable agents for administration.

In some embodiments, the cannabinoids are formulated in accordance with routine procedures as a pharmaceutical composition adapted for intravenous, subcutaneous or parenteral administration to human beings. In some embodiments, compositions for administration are solutions in sterile isotonic aqueous buffer. Where necessary, the composition can also include a solubilizing agent. Generally, the ingredients are supplied either separately or mixed together in unit dosage form, for example, as a dry lyophilized powder or water free concentrate in a hermetically sealed container such as an ampoule indicating the quantity of active agent. In some embodiments, an ampoule of sterile water for injection or saline can be provided so that the ingredients can be mixed prior to administration.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The cannabinoids can also be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In certain embodiments, the compositions are pharmaceutical compositions. In some embodiments, formulations are prepared for storage and use by combining the active agents with a pharmaceutically acceptable vehicle (e.g. carrier, excipient) (Remington, The Science and Practice of Pharmacy 20th Edition Mack Publishing, 2000). In some embodiments, pharmaceutical compositions of the present invention are characterized as being at least sterile and pyrogen-free. As used herein, “pharmaceutical formulations” include formulations for human and veterinary use. Pharmaceutical compositions of the invention can be packaged for use in liquid form, or can be lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

For a broad overview of controlled delivery systems, see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nano spheres, and nanoparticles. Microcapsules can contain the therapeutically active agents as a central core. In microspheres the therapeutic can be dispersed throughout the particle. Particles, microspheres, and microcapsules smaller than about 1 μm are generally referred to as nanoparticles, nanospheres, and nanocapsules, respectively. Microparticles are typically around 100 μm in diameter. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

In some embodiments, polymers can be used for controlled release of compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). In yet another aspect, liposomes can be used for controlled release as well as drug targeting of the lipid-capsulated drug (Betageri et al., Liposome Drug Delivery Systems, Technomic Publishing Co., Inc., Lancaster, Pa. (1993)).

In some embodiments, the pharmaceutical composition comprises an effective amount of one or more cannabinoids, at least one surfactant, at least one co-solvent; and water; wherein said cannabinoid composition yields the cannabinoid preparation capable of having increased bioavailability. In another aspect, the at least one surfactant comprises one or more, or alternatively, at least two surfactants. Further, the one or more surfactants have an HLB in the range of 1 to 8 and form a self-assembling emulsion. In another embodiment, at least one surfactant comprises an HLB in the range of 9-20. In yet another embodiment, the at least one surfactant is selected from a group consisting of: oleic acid, sunflower oil, lecithin, phosphatidylcholine, isopropyl myristate, stearic acid, medium and long chain triglycerides, Labrasol, polysorbate 20, polysorbate 80, or other ethoxylated surfactant, and sorbitan trioleate, or other sorbitan surfactant. In some embodiments, the at least one co-solvent is selected from a group consisting of: methanol, ethanol, isopropyl alcohol, butanol, pentanol, hexanol, ethylene glycol, propylene glycol, dipropylene glycol, glycerol, erythritol, xylitol, mannitol, sorbitol, diethylene glycol monoethyl ether (Transcutol), any derivative thereof, and combinations relating thereto. In some embodiments, the cannabinoid preparation of the present disclosure is encapsulated by surfactants to form micelles having uni-, bi-, or multi-lamellar structures.

It is a further object of the present disclosure to provide a composition capable of transdermal delivery. In one aspect, a cannabinoid composition is provided that is capable of transdermal delivery, comprising: a cannabinoid composition further comprising: a cannabinoid preparation ranging from 0.1% to 10% (w/w); at least one surfactant ranging from 0.1% to 25% (w/w); at least one co-solvent ranging from 10% to 60% (w/w); water ranging from 16% to 60% (w/w); a stabilizer ranging from 0.01% to 3% (w/w).

In some embodiments, the compositions comprises a stabilizer in the range of 0.01% to 3% (w/w). In some embodiments, the composition comprises a thickening agent in the range of 0.01% to 5% (w/w). In some embodiments, the composition comprises terpenes in the range of 0.1% to 5% (w/w). In some embodiments, the total cannabinoid preparation ranges from 0.01% to 20% (w/w), the at least one surfactants range from 5% to 50% (w/w), the at least one co-solvents range from 10% to 70% (w/w), the water ranges from 16% to 60% (w/w), and the stabilizer ranges from 0.01% to 3% (w/w).

In some embodiments, the cannabinoid preparation is encapsulated by surfactants to form micelles having uni-, bi-, or multi-lamellar structures. The cannabinoid composition may further comprise a stabilizer in the range of 0.01% to 3% (w/w). The cannabinoid composition may further comprise a thickening agent in the range of 0.01% to 5% (w/w). The cannabinoid composition may even further comprise terpenes in the range of 0.1% to 5% (w/w). In another aspect the cannabinoid preparation ranges from 0.1% to 20% (w/w), the at least one surfactants range from 5% to 50% (w/w), the at least one co-solvents range from 5% to 70% (w/w), the water ranges from 16% to 60% (w/w), and the stabilizer ranges from 0.01% to 3% (w/w).

In some embodiments, the composition comprises: a cannabinoid composition further comprising a cannabinoid preparation ranging from 0.1% to 10% (w/w), at least one surfactant ranging from 0.1% to 25% (w/w), at least one co-solvent ranging from 10% to 70% (w/w), water ranging from 16% to 60% (w/w), and a stabilizer ranging from 0.01% to 3% (w/w).

In some embodiments, the composition comprises one or more cannabinoids in complex with one or more cyclodextrins. Cyclodextrins are cyclic oligosaccharides of (alpha-1,4)-linked alpha-D-glucopyranose units, with a lipophilic central cavity and a hydrophilic outer surface. As a result of their molecular structure and shape, they can act as molecular containers by trapping drugs or other molecules in their internal cavity. No covalent bonds are formed or broken during drug cyclodextrin complex formation, and in aqueous solution, the complexes readily dissociate and free drug molecules remain in equilibrium with the molecules bound within the cyclodextrin cavity (see, Tiwari et al (2010) Cyclodextrins in delivery systems: Applications. J. Pharm. Bioallied Sci. 2:72-79). Derivatives of cyclodextrins that are hydroxypropyl (HP), methyl (M) and sulfobutylether (SBE) substituents are useful as pharmaceutical excipients.

Cyclodextrins for use, for example, in cannabinoid/cyclodextrin complex, include beta-cyclodextrins such as hydroxypropyl-beta-cyclodextrin, sulfobutylether-beta-cyclodextrin, maltoxyl-beta-cyclodextrin, and methylated cyclodextrins. Encompassed are alpha-cyclodextrins (6 glucopyranose units), beta-cyclodextrins (7 glucopyranose units), and gamma-cyclodextrins (8 glucopyranose units). Methylated cyclodextrins can improve aqueous solubility, dissolution rate, and bioavailability of cannabinoids.

In some embodiments, the composition comprises one or more cannabinoids and one or more solubilizers such as detergents, surfactants, organic solvents, and chaotropic agents. These can be one or more of, polyethylene glycol (PEG), propylene glycol, dibutyl subacetate, glycerol, diethyl phthalate (phthalate esters), triacetin, citrate esters-triethyl citrate, acetyltriethyl citrate, tributyl citrate, acetyltributyl citrate, benzyl benzoate, sorbitol, xylitol, bis(2-ethyllhexyl) adipate, mineral oil, polyhydric alcohols such as glycerin and sorbitol, glycerol esters such as glycerol, triacetate; fatty acid triglycerides, polyoxyethylene sorbitan, fatty acid esters such as TWEENS, polyoxyethylene monoalkyl ethers such as BRIJ series and MYRJ series, sucrose monoesters, lanolin esters, lanolin ethers.

In some embodiments, the surfactant can include sorbitan trioleate, sorbitan monooleate, sorbitan monolaurate, polyoxyethylene (20) sorbitan monolaurate, polyoxyethylene (20) sorbitan monooleate, oleyl polyoxytheylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, isopropyl palmitate, glyceryl monooleate, glyceryl monostearate, glyceryl monoricinoleate, cetyl alcohol, stearyl alcohol, cetyl pyridinium chloride, olive oil, glyceryl monolaurate, corn oil, cotton seed oil, and sunflower seed oil.

The present disclosure can include formulations that contain a buffer with a pKa, as measured at room temperature, such as boric acid (pKa 9.2), CHES (pKa 9.5), bicine (pKa 8.3), HEPES (pKa 7.5), MES (pKa 6.1), MOPS (pKa 7.2), PIPES (pKa 6.8), Tris (pKa 8.1), imidazole (pKa 6.9), glycine (pKa 2.3), acetate (pKa 4.7), citrate (pKa 6.4), phosphate (pKa 7.21, 2.16, 12.32), malate (pKa 5.13), cacodylate (pKa 6.27), and the like. Without regard to any buffer, the present disclosure provides a formulation, or provides a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like.

In some embodiments, the compositions comprise aqueous micelle suspensions of one or more cannabinoids. In some embodiments, the micelles comprise phospholipids and/or cholesterol. In some embodiments. the average micelle diameter size is in a range between 50 and 1000 nm. In some embodiments, the stable aqueous micelle suspension of one or more cannabinoids further comprises a stabilizer selected from the group consisting of guar gum, xyanthan gum cellulose hyaluronic acid, polyvinyl pyrrolidone (PVP), alginate, chondritin sulfate, poly gamma glutamic acid, gelatin, chitisin, corn starch and flour, in an amount from about 0.25% to about 2% (w/v).

In some embodiments, the compositions comprise a liposomal formulation of one or more cannabinoids. In some embodiments, the average diameter size of liposomes in the inventive composition is from about 200 to about 400 nm. In some embodiments, the maximum phospholipid content of the hydrophobic/lipophilic membrane is 50% of the total composition and the hydrophobic/lipophilic membrane comprises about 26% phosphatidylcholine, about 10% phosphatidylethanolamine, about 13% phosphonophospholipids, and about 1% of other phospholipids.

In another embodiment, the invention provides a microneedle device (e.g., as described herein) comprising a composition comprising an effective amount of one or more cannabinoids. In some embodiments, the microneedle device comprises a composition comprising one or more cannabinoids in combination with one or more active agents, for example, as described herein.

In another embodiment, the invention provides a kit comprising a microneedle device (e.g., as described herein) comprising an effective amount of one or more cannabinoids, and optionally instructions for use of the device. In some embodiments, the kit comprises one or more solvents for dissolving the cannabinoid composition prior to use.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

I claim:

1. A method for treating a condition or disease in a subject, comprising administering to the subject's skin a composition comprising an effective amount of one or more cannabinoids, wherein the composition is administered with a microneedle delivery device.

2. The method of any one of claim 1, wherein the microneedle delivery device comprises
i) one or more microneedles, wherein the microneedles are hollow or non-hollow, wherein one or multiple grooves are inset along an outer wall of the microneedles; and
ii) a reservoir that holds the composition to be delivered, wherein the reservoir is attached to or contains a means to encourage flow of the composition contained in the reservoir into the skin;
wherein the administering comprises a repeated motion of penetrating the microneedle delivery device into the skin of the subject,
wherein the composition is delivered into the skin by passing through the one or multiple grooves along the outer wall of the microneedle.

3. The method of claim 2, wherein the microneedles are non-hollow.

4. The method of any of claims 1-3, wherein the means to encourage flow of the composition contained in the reservoir into the skin is selected from the group consisting of a plunger, pump and suction mechanism.

5. The method of claim 4, wherein the means to encourage flow of the composition contained in the reservoir into the skin is a mechanical spring loaded pump system.

6. The method of any of claims 1-5. wherein the microneedles have a single groove inset along the outer wall of the microneedle, wherein the single groove has a screw thread shape going clockwise or counterclockwise around the microneedle.

7. The method of any of claims 1-6, wherein the microneedles are from 0.1 mm to about 2.5 mm in length and from 0.01 mm to about 0.05 mm in diameter.

8. The method of any of claims 1-7, wherein the microneedles are made from a substance comprising gold.

9. The method of any of claims 2-8, wherein the plurality of microneedles comprises an array of microneedles in the shape of a circle.

10. The method of any of claims 1-9, wherein the microneedles are made of 24-carat gold plated stainless steel and comprise an array of 20 microneedles.

11. The method of any of claims 1-10, wherein the one or more cannabinoids are selected from cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and mixtures of cannabinoids.

12. The method of any of claims 1-11, wherein the one or more cannabinoids are administered with an effective amount of an antineoplastic agent.

13. The method of claim 12, wherein the antineoplastic agent is selected from the group consisting of alkylating agents, platinum compounds (e.g., cisplatin, carboplatin, oxaliplatin, mechlorethamine, cyclophosphamide, chlorambucil, ifosfamide), anti-metabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins and combinations thereof.

14. The method of claim 12, wherein the antineoplastic agent is selected from the group consisting of LDE225B, vismodegib, RIVEDGE, PD-L1, Patidegib, Nivolumab, Nivolumab+Ipilimumab, Imiquimod, Metvix PDT, Diclofenac, Diclofenac+Calcitriol, Calcitriol, Methylaminolevulinate, Fractionated 5-aminolevulinic acid hydrochloride, PEP005, SUB A-Itraconazole, methyl-aminolevulinatem, Remetinostat, verteporfin PDT, Sonidegib, Itraconazole, vismodegib, Picato, Resiquimod, API 31510, Aminolevulinic acid, arsenic trioxide, Patidegib, REGN2810, Buparlisib, Oshadi D & Oshadi R, Celecoxib, Tazarotene, Sinecatechins 10%, aminolevulinic acid hydrochloride, Vismodegib, FOLFOX, FOLFIRI, Bevacizumab, Tazarotene, Hexylaminolevulinate, Aminolevulinic Acid, Nano Emulsion, Methylaminolevulinate, liposomal T4N5 lotion, Carboplatin, Cyclophosphamide, Etoposide, Methotrexate, Vincristine Sulfate, Acetylcysteine, Bevacizumab, Eflornithine, Celecoxib, erlotinib hydrochloride, Poly-ICLC, aminolevulinic acid, eflornithine, triamcinolone, 18F-fludeoxyglucose (18F-FDG), 18F-FPPRGD2, Fluconazole, cevimeline hydrochloride, megestrol acetate, Amifostine, Carboplatin, Etoposide, Ifosfamide, Hypericum perforatum, Docetaxel, Nicotinamide, doxepin hydrochloride, capecitabine, oxaliplatin, DetoxPC, Capecitabine, Carboplatin, epirubicin hydrochloride, cisplatin, paclitaxel, Tretinoin, doxorubicin hydrochloride, gemcitabine hydrochloride, indinavir sulfate, ritonavir, 5-fluorouracil, and combinations thereof.

15. The method of claim 12, wherein the antineoplastic agent is selected from the group consisting of Imiquimod, VDA-1102, Fluorouracil, Cetaphil, SOR007 (Uncoated Nanoparticulate Paclitaxel), Aminolevulinic Acid, PEP005 (ingenol mebutate), ingenol disoxate, Ingenol Mebutate, Aminolevulinic Acid (ALA), Biafine, Polysporin, 5-FU, A-101 Solution (High-Concentration Hydrogen Peroxide), lidamycin phosphate and benzoyl peroxide 1.2%/3.75% combination, Eflornithine, Triamcinolone, Polyphenon E, BLU-U, ACT01, Levulan®, Kerastick®, perillyl alcohol, aminolevulinic acid, liposomal T4N5, Aminolevulinic Acid, GDC 695, Diclofenac Sodium, KX2-391, ingenol disoxate, ingenol disoxate, Celecoxib, Bupivacaine+Clonidine, Secukinumab, Pimecrolimus and combinations thereof.

16. The method of any of claims 1-15, wherein the disease or condition is selected from the group consisting of anorexia, emesis, sunburn, photodamaged skin, acne, psoriasis, atopic dermatitis, pain, inflammation, multiple sclerosis, neurodegenerative disorders (e.g., Parkinson's disease, Huntington's disease, Tourette's syndrome, Alzheimer's disease), epilepsy, glaucoma, osteoporosis, schizophrenia, cardiovascular disorders, cancer, actinic keratosis, obesity, amyotrophic lateral sclerosis (ALS), atherosclerosis, chronic pain, diabetes mellitus, dystonia, fibromyalgia, gastrointestinal disorders, gliomas, cancer, Hepatitis C, human immunodeficiency virus (HIV), HIV dementia, Huntington Disease hypertension, incontinence, methicillin-resistant Staphylococcus aureus (MRSA), post-traumatic stress disorders (PTSD), pruritus, rheumatoid arthritis, sleep apnea, and metabolic syndrome-related disorders.

17. The method of any of claims 1-16, further comprising administering to the subject one or more additional therapies.

18. The method of claim 16, wherein the additional therapy includes radiation, surgery, chemotherapy, simple excision, Mohs micrographic surgery, Curettage and electrodesiccation, cryosurgery, photodynamic therapy, topical chemotherapy, and topical immunotherapy.

19. A microneedle drug delivery device comprising a composition comprising an effective amount of one or more cannabinoids.

20. The microneedle drug delivery device of claim 19, wherein the one or more cannabinoids are in lyophilized or powder form.
